(19) United States
(12) Patent Application Publication
(10) Pub. No.: US 2010/0198205 A1
(43) Pub. Date: Aug. 5, 2010

(54) Low Pressure Liquid Nitrogen Cryosurgical System

(75) Inventors: James B. Ross, Pleasanton, CA (US); Russell L. DeLonzor, Pleasanton, CA (US); Mathew J. Nalipinski, Pleasanton, CA (US); Keith Turner, Cambridge (GB); David J. Foster, Cambridge (GB); Samuel C. Richards, Cambridge (GB); Michael R. Cane, Cambridge (GB)

Correspondence Address: CROCKETT & CROCKETT, P.C., 26020 ACERO, SUITE 200, MISSION VIEJO, CA, 92691, US

(73) Assignee: Sanarus Technologies, LLC

(21) Appl. No.: 12/693,962

(22) Filed: Jan. 26, 2010

Related U.S. Application Data
(63) Continuation of application No. 11/317,579, filed on Dec. 23, 2005, now abandoned.

(51) Int. Cl. A61B 18/02

(52) U.S. Cl. 606/21

(57) ABSTRACT

A cryosurgical system using a low-pressure liquid nitrogen supply, which requires only 0.5 to 1 bar of pressure to provide adequate cooling power for treatment of typical breast lesions. The pressure may be provided by supplying lightly pressurized air into the dewar, by heating a small portion of the nitrogen in the dewar, or with a small low pressure pump.

This application is a continuation application of U.S. application Ser. No. 11/317,579, filed Dec. 23, 2005.

### FIELD OF THE INVENTIONS

The inventions described below relate the field of cryosurgical systems.

### BACKGROUND OF THE INVENTIONS

Cryosurgery refers to the freezing of body tissue in order to destroy diseased tissue. Minimally invasive cryosurgical systems generally include a long, slender cryoprobe adapted for insertion into the body so that the tip resides in the diseased tissue, and source of cryogenic fluid, and the necessary tubing to conduct the cryogenic fluid into and out of the probe. These cryosurgical systems also include heating systems, so that the probes can be warmed to enhance the destructive effect of the cryoablation and to provide for quick release of the cryoprobes when ablation is complete.

Our own Visica® cryoablation system has proven effective for the treatment of lesions within the breast of female patients. The system uses Joule-Thompson cryoprobes, and uses argon gas as the cryogenic fluid. The argon gas, supplied at room temperature but very high pressure, expands and cools within the tip of the cryoprobe to generate the cooling power needed to freeze body tissue to cryogenic temperatures. The Visica® cryoablation system uses high-pressure helium flow through the cryoprobe to heat the probe. The system requires large supplies of argon gas, but is otherwise quite convenient.

Earlier cryoprobes proposed for other surgeries, such as prostrate cryosurgery, used liquid nitrogen, which has the advantage that is more readily available than argon, and the volume necessary for a given cryosurgical procedure is much smaller then argon. Cryoablation systems using liquid nitrogen, such as the Accuprobe™ cryoablation system, have been proposed and used, but these systems have been abandoned in favor of the gas Joule-Thompson systems. The literature and patent filings indicate that liquid nitrogen systems were plagued by various problems, such as vapor lock and excessive consumption of liquid nitrogen. Proposals to solve these problems, though never successfully implemented, include various schemes to prevent vapor lock and maximize efficiency of the heat exchange. See Rubinsky, et al., Cryosurgical System For Destroying Tumors By Freezing, U.S. Pat. No. 5,334,181 (Aug. 2, 1994) and Rubinsky, et al., Cryosurgical Instrument And System And Method Of Cryosurgery, U.S. Pat. No. 5,674,218 (Oct. 7, 1997), and Littrup, et al., Cryotherapy Probe and System, PCT Pub. WO 2004/064914 (Aug. 5, 2004).

To date, the problems inherent in liquid nitrogen systems have led the art to avoid them in favor of gaseous argon systems.

### SUMMARY

The devices and methods described below provide for use of liquid nitrogen in cryoablation systems while preventing the vapor lock typically associated with those systems, and minimizing the amount of liquid nitrogen used in a given procedure. The system uses cryoprobes of coaxial structure, and is supplied with cryogen from a dewar of liquid nitrogen. The system includes various enhancements to avoid heat transfer from the liquid nitrogen to the system components, and as a result permits use of very low-pressure nitrogen, and, vice-versa, the use of low pressure nitrogen permits use of the various enhancements (which could not be used in a high pressure system). The result is a system that provides sufficient cooling power to effectively ablate lesions, tumors and masses within the breast of female patients while using very little nitrogen and a compact and inexpensive system based on readily available and easy to handle liquid nitrogen.

The system includes a low-pressure liquid nitrogen supply, which requires only 0.5 to 1 bar of pressure to provide adequate cooling power for treatment of typical breast lesions. The pressure may be provided by supplying lightly pressurized air into the dewar, by heating a small portion of the nitrogen in the dewar, or with a small low pressure pump. The use of low pressure liquid nitrogen permits use of polymers for several components, such as the supply hose, the cryoprobe inlet tube, and various hose connectors which are typically made of metal, so that the system is much more efficient and uses very little liquid nitrogen. Additionally, because the liquid nitrogen is lightly pressurized, the boiling point remains low, and the liquid temperature also remains low compared with higher pressure systems.

### BRIEF DESCRIPTION OF THE DRAWINGS

FIG. 1 illustrates a cryosurgical system which uses liquid nitrogen as a cryogen.

FIG. 2 illustrates a supply hose modified to enhance operation of the system of FIG. 1.

FIG. 3 illustrates a cryosurgical system which uses liquid nitrogen as a cryogen and a small heater in the cryogen source to pressurize the cryogen.

FIG. 4 illustrates a cryosurgical system which uses liquid nitrogen as a cryogen and a pump for driving cryogen flow.

### DETAILED DESCRIPTION OF THE INVENTIONS

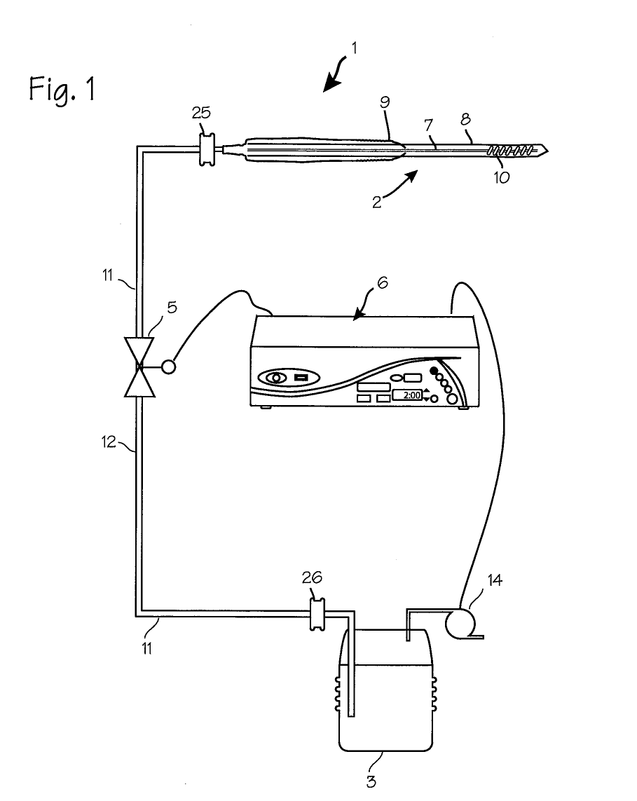

FIG. 1 illustrates a cryosurgical system which uses liquid nitrogen as a cryogen. The cryosurgical system 1 comprises cryoprobe 2, a cryogen source 3, pressurization pump 4, flow control valve 5 interposed between the cryogen source and the cryoprobe, and a control system 6 for controlling the control valve. The system may be adapted to accommodate multiple cryoprobes with the addition of appropriate manifolds, and the control system may be computer-based or otherwise operable to automatically control the control valves and other system components to effect the cooling profiles for desired cryosurgeries. The desired flow of cryogen from the dewar to the cryoprobe is induced in this embodiment by pressurizing the cryogen source with air delivered by the pressurization pump.

The cryoprobe 2 comprises an inlet tube 7, a closed-ended outer tube 8, and a handle portion 9. The inlet tube comprises a small diameter tube, and the outer tube comprises a closed end tube, disposed coaxially about the inlet tube. The inlet tube is preferably a rigid tube with low thermal conductivity, such as polyetheretherketone (PEEK, which is well know for its high temperature performance), fluorinated ethylene propylene (FEP) or polytetrafluoroethylene. The cryoprobe preferably includes the flow-directing coil 10 disposed coaxially between the inlet tube and the outer tube at the distal end of the cryoprobe. The flow directing coil serves to direct flow onto the inner surface of the outer tube, thereby enhancing heat transfer from the outer tube that the cryogen fluid stream. The cryoprobe is describe in detail in our co-pending application, DeLonzor, et al., Cryoprobe For Low Pressure Systems, Attorney Docket No. 212/801 filed Dec. 23, 2005, the entirety of which is hereby incorporated by reference. The cryoprobe is supplied with cryogen through the supply hose 11, described in detail below, the control valve 5, and the dewar outlet hose 12. When used in the current system, with low-pressure liquid nitrogen, the cryoprobe having an inlet tube of 1 mm inner diameter and 1.6 mm outer diameter, and an outer tube with 2.4 mm inner diameter and 2.7 mm outer diameter works well. Probes with outer diameters of up to 4 mm and down to 1.5 mm

The cryogen source is preferable a dewar of liquid nitrogen. The dewar may comprise a composite material of low thermal conductivity, and is preferably fitted with a low pressure relief valve set to lift at about 20 to 30 psi. The dewar is lightly pressurized, in the range of about 0.5 to 1 bar over ambient pressure (about 7.25 to 14.5 psi), with air or other suitable gas, through compressor 13. Any other means of pressurizing the liquid nitrogen may be used, including use of a pump at the outlet of the dewar, heating a small portion of the liquid nitrogen or gaseous nitrogen in the dewar to boost pressure in the dewar, or heating the liquid nitrogen at the exit of the dewar.

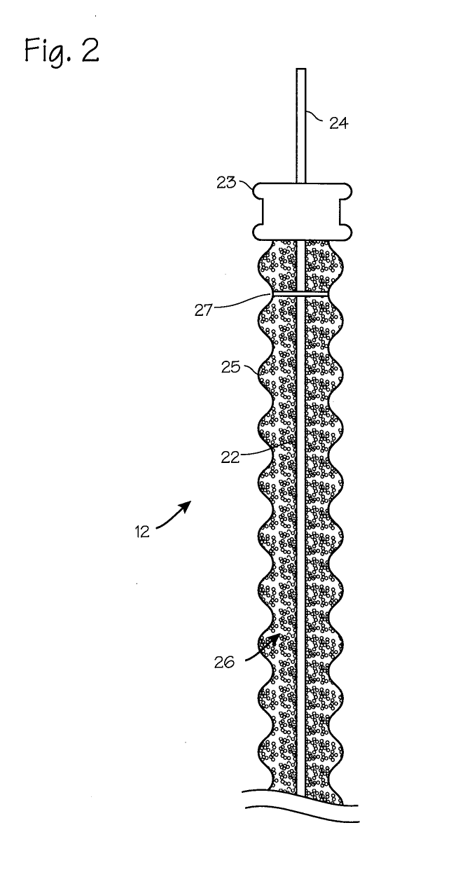

The supply hose 11, illustrated in cross section in FIG. 2, is particularly suited to use with the low-pressure liquid nitrogen system. The supply hose comprises an inner tube 21 of FEP, nylon or other thermally resistant polymer with very low thermal mass (the ability to absorb heat) (polymers typically have a low coefficient of thermal conductivity, about 0.2 to 0.3 W/mK) which remains flexible at cryogenic temperatures of the liquid nitrogen. The outer tube 22 of any suitable flexible material (ethylene vinyl acetate (EVA), low density polyethylene (LDPE), or nylon, for example) which is corrugated transversely to promote omni-directional flexibility. The space between the inner tube and outer jacket is filled with aerogel beads or particles (indicated at item 23) or provided as a continuous tube of aerogel. (Aerogel refers to a synthetic amorphous silica gel foam, with a very low thermal conductivity (10⁻³ W/mK and below) with pores sizes in the range of about 5 to 100 nm.) The supply hose is preferably about 6 feet (2 meters) long, which provides convenient working length while minimizing cooling losses. The outer tube is preferably about 15 mm in outer diameter, while the inner tube is preferably about 1 mm in inner diameter and 1.5 mm outer diameter. The aerogel beads, if used, may be about 1 mm diameter beads, and may be wetted lightly with silicone oil or similar clumping agent to prevent excessive dust dispersion in the case of rupture of the inner tube and/or outer jacket. Occasional spacers, in the form of washers 24 comprising materials such as polymethacrylimide closed-cell foam (PMI), may be placed along the inner tube to prevent collapse of the outer jacket and displacement of the aerogel beads. An aerogel tube may be formed by wrapping flexible aerogel blankets around the inner tube, or extruding and aerogel and binder mixture. The annular space between the inner tube and outer jacket of the supply hose may also be filled with other low thermal mass materials such as perlite powder, cotton fiber, etc., though aerogel has proven particularly effective in limiting warming of the cryogen within the supply tube while providing a supply hose that is easy to manipulate during the course of a cryosurgical procedure. The dewar outlet hose 12 may be constructed in the same fashion as the supply hose 11, though a typical unwieldy vacuum insulated cryogen hose may suffice depending on the expected heat losses, heat load on the cryoprobe and cryogenic flow rates. Coupling 25 is provided to releasably attach the supply hose to the cryoprobe, so that the supply hose can readily be attached and detached from the cryoprobe without use of special tools. Because the system operates at low pressure, the coupling may be composed substantially, if not entirely, of a polymer such as nylon, so that the thermal mass and thermal conductivity of the couplings are very low and the cooling power of the cryogen will not be wasted in cooling the couplings. Couplings elsewhere in the system may also be comprised of polymers and similar materials with low thermal conductivity, such as the coupling 26 at the dewar outlet. The couplings may comprise any releasable fitting structure, such as Luer fittings, bayonet fittings, large threaded fitting that are operable by hand, quick-lock fittings and the like.

In use, the cryoprobe is inserted into the body, with its distal tip within a lesion or other diseased tissue that is to be ablated, the surgeon will operate the systems through controls on the control system 6. The dewar is pressurized to about 0.5 to 1 bar (about 7.25 to 14.5 psi). The control valve is operated to provide flow to the cryoprobe at about 0.5 to 2 grams per second to effect cryoablation of the lesion. The flow of cryogen is continued as necessary to freeze the lesion to cryogenic temperatures. Preferably the operation of the system is controlled automatically via the control system, though it may be implemented manually by a surgeon, including manual operation pressurizing means of the dewar and manual operation of the control valve. When used to treat lesions in the breast, the system may be operated according to the parameters described in our U.S. Pat. No. 6,789,545.

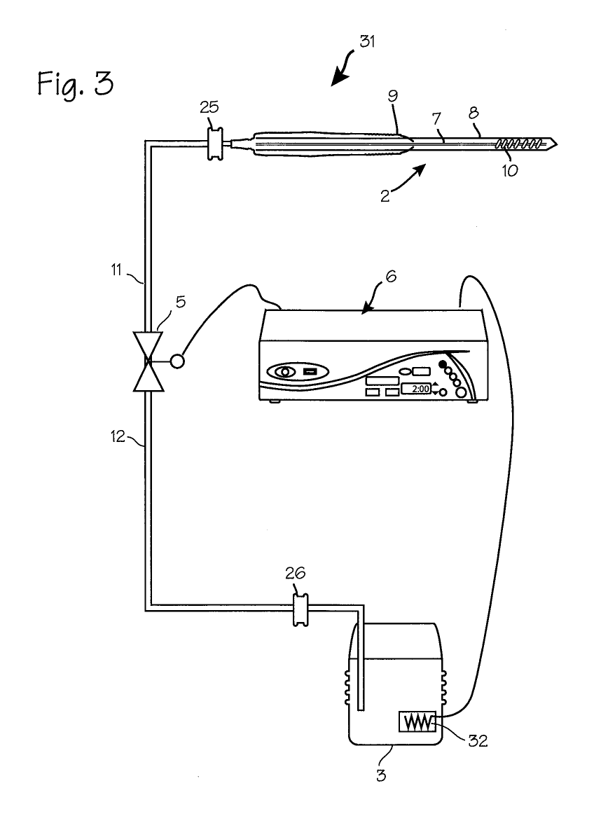

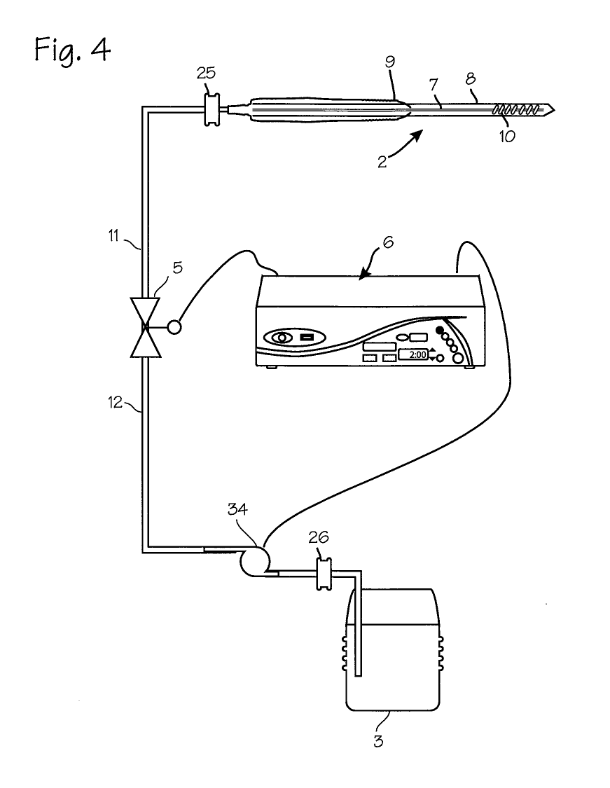

FIG. 3 illustrates a liquid nitrogen cryosurgical system 31 which uses a heater to generate the desired pressure to drive the system. This system includes the cryoprobe 2, cryogen source 3, control valve 5 and control system 6 of FIG. 1. A heater 32 is provided in the dewar, and is operable to heat a small volume of the nitrogen in the dewar and thereby increase the pressure in the dewar to the desired level of 0.5 to 1 bar (7.25 to 14.5 psi) above ambient pressure. The control system can automatically control the heater with feedback from pressure sensors in the dewar. The heater may be submersed in the liquid nitrogen or placed within the gas above the liquid, and it may be disposed on the inside wall of the dewar or suspended within the dewar. As shown in FIG. 4, the necessary pressure may also be provided with a cryogenic pump 33 (though this entails significant additional cost of a cryogenic pump). In FIG. 4, a cryogenic pump is placed at the outlet of the dewar, in line with the dewar outlet hose 12, and is operable by the control system to provide liquid nitrogen at about 5. to 1 bar of pressure to the control valve and cryoprobe. The use of air, as shown in FIG. 1, and the use of the heater as shown in FIG. 3, both entail addition of heat to the dewar system, but this has proven acceptable given the additional thermal gains obtained by the various components described above.

The systems described above may be employed with various liquid cryogens, though liquid nitrogen is favored for is universal availability and ease of use. Also, though system has been developed for use in treatment of breast disease, it may be employed to treat lesions elsewhere in the body. Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A cryosurgical system comprising:
a cryoprobe comprising a closed-ended outer tube and an inlet tube disposed within the outer tube, said cryoprobe having a distal end corresponding to the closed end of the outer tube which is adapted for insertion into the body of a patient and a proximal end adapted for connection to a source of cryogenic liquid;
a source of cryogenic liquid and pressurizing means for pressuring the cryogenic liquid;
a valve in fluid communication between the source and the cryoprobe for controlling flow of cryogenic liquid from the source to the cryoprobe;
a supply hose connecting the valve to the proximal end of the cryoprobe and establishing a liquid flow path from the valve to the inlet tube of the cryoprobe;
a control system operable to control the pressurizing means and valve to provide cryogenic liquid to the cryoprobe, said control system being programmed to control the pressurizing means to pressurize the source in the range of about 0.5 to 1 bar, and control the valve to provide cryogenic fluid flow to the cryoprobe and
a flow directing coil disposed between the inlet tube and the outer tube, at a distal end of the cryoprobe.

2. A cryosurgical system of claim 1 wherein the supply hose comprises:
an inner tube comprising a polymer, said inner tube having an inner diameter of about 1 mm and an outer jacket comprising a polymer, said outer jacket having a diameter of about 15 mm; with the space between inner tube and outer jacket being filled with aerogel.

3. A cryosurgical system of claim 2 further comprising:
means for releasably attaching the cryoprobe proximal end to the supply hose, said means comprising low thermal mass polymeric fittings.

4. A cryosurgical system of claim 1 wherein the pressurizing means comprises:
a pump operably connected to the source to pump air into the source and thereby pressurize the source to about 0.5 to 1 bar of pressure.

5. A cryosurgical system of claim 1 wherein the pressurizing means comprises:
a heater in thermal communication with the cryogen in the source, said heater being operable to heat a small volume of the cryogen and thereby pressurize the source to about 0.5 to 1 bar of pressure.

6. A cryosurgical system of claim 1 wherein the pressurizing means comprises:
a pump operably connected to the source to pump cryogen from the source to the valve at a pressure of about 0.5 to 1 bar of pressure.

7. A method of cryoablating diseased body tissue in a patient, said method comprising:
providing a cryosurgical system comprising:
a cryoprobe comprising a closed-ended outer tube and an inlet tube disposed within the outer tube, said cryoprobe having a distal end corresponding to the closed end of the outer tube which is adapted for insertion into the body of a patient and a proximal end adapted for connection to a source of cryogenic liquid;
a source of cryogenic liquid and pressurizing means for pressuring the cryogenic liquid;
a valve in fluid communication between the source and the cryoprobe for controlling flow of cryogenic liquid from the source to the cryoprobe;
a supply hose connecting the valve to the proximal end of the cryoprobe and establishing a liquid flow path from the valve to the inlet tube of the cryoprobe;
a control system operable to control the pressurizing means and valve to provide cryogenic liquid to the cryoprobe, said control system being programmed to control the pressurizing means to pressurized the source in the range of about 0.5 to 1 bar, and control the valve to provide cryogenic fluid flow to the cryoprobe;
a flow directing coil disposed between the inlet tube and the outer tube, at a distal end of the cryoprobe;
operating the control system to pressurize the source to about 0.5 to 1 bar of pressure and operating the control valve to provide cryogenic liquid flow to the cryoprobe, thereby providing about 0.5 to 2 grams per second of cryogenic liquid to the cryoprobe;
continuing the flow of cryogen as necessary to freeze the diseased tissue to cryogenic temperatures.
